(19) United States
(12) Patent Application Publication
(10) Pub. No.: US 2018/0235924 A1
(43) Pub. Date: Aug. 23, 2018

(54) COMPOSITIONS FOR TOPICAL APPLICATION COMPRISING BENZOYL PEROXIDE AND ADAPALENE

(71) Applicant: SOL-GEL TECHNOLOGIES LTD., Ness Ziona (IL)

(72) Inventors: Ofer TOLEDANO, Kfar Saba (IL); Alon SERI-LEVY, Rehovot (IL); Karine NEIMANN, Ness Ziona (IL); Ori NOV, Tarum (IL)

(73) Assignee: SOL-GEL TECHNOLOGIES LTD., Ness Ziona (IL)

(21) Appl. No.: 15/753,397

(22) PCT Filed: Aug. 17, 2016
(86) PCT No.: PCT/IL2016/050895

§ 371 Date: Feb. 19, 2018

Related U.S. Application Data
(60) Provisional application No. 62/207,452, filed on Aug. 20, 2015.

(51) Int. Cl. A61K 31/327; A61K 9/00; A61K 9/16; A61P 17/06; A61P 17/04; A61P 17/10

(52) U.S. Cl. CPC A61K 31/327; A61K 9/0014; A61K 9/16; A61K 31/192; A61P 17/04; A61P 17/10; A61K 2300/00; A61P 17/06

(57) ABSTRACT

The invention provides compositions comprising as active agents benzoyl peroxide (BPO), adapalene and optionally clindamycin phosphate, kits and uses thereof in methods for treating skin surface condition in a subject in need thereof.

This application is a Continuation-in-Part of International Application Number PCT/IL2016/050895, filed Aug. 17, 2016, which claims priority of U.S. Ser. No. 62/207,452, filed Aug. 20, 2015, each of which is hereby incorporated by reference in its entirety.

### FIELD OF THE INVENTION

This invention relates to compositions comprising BPO, adapalene and optionally clindamycin phosphate for topical application.

### BACKGROUND OF THE INVENTION

Nearly 17 million people in the United States have acne, making it the most common skin disease. Although acne is not a serious health threat, severe acne can lead to disfiguring, permanent scarring, which can be upsetting for people who suffer from the disorder.

Two of the most commonly used ingredients in topical treatments are Benzoyl Peroxide (BPO) and all trans Retinoic acid (Tretinoin (ATRA)) which can be very effective in treating mild to moderate cases of non-inflammatory acne. Benzoyl peroxide acts by destroying P. acnes, the bacteria that causes the condition acne. It acts as an antiseptic and as an oxidizing agent, reducing the number of comedones, or blocked pores. Tretinoin (ATRA) is a unique topical medication used in the treatment of acne that allows the keratin plugs of microcomedones to be expelled, thus fewer lesions are able to rupture and cause papules, pustules and nodules of inflammatory acne. A combination drug of BPO and ATRA should have both comedogenesis and bacteriostatic effect in acne treatment. However, two main obstacles to such combination is instability of ATRA in presence of BPO and severe adverse events such as erythema, irritation, buming, stinging, scaling and itching.

Compositions and methods for treatment acne comprising BPO and/or a Retinoid are described for example in U.S. Pat. No. 4,350,681, U.S. Pat. No. 4,361,584, U.S. Pat. No. 4,387,107, U.S. Pat. No. 4,497,794. U.S. Pat. No. 4,671,956, U.S. Pat. No. 4,960,772. U.S. Pat. Nos. 5,086,075, 5,145,675, U.S. Pat. No. 5,466,446, U.S. Pat. No. 5,632,996, U.S. Pat. No. 5,767,098, U.S. Pat. No. 5,851,538, U.S. Pat. Nos. 5,955,109, 5,879,716, 5,955,109 U.S. Pat. No. 5,998,392, U.S. Pat. No. 6,013,637, U.S. Pat. No. 6,117,843, Pub. No. US 2003/0170196, US2002064541, and 20050037087. H. Tatapudy et al., Indian Drugs, 32(6), 239-248, 1995, describes benzoyl peroxide microcapsules, prepared by coacervation phase separation process.

Clindamycin phosphate is a topical antibiotic approved for the treatment of acne. This drug belongs to the class of antibiotics known as lincosamides. By inhibiting bacteria protein synthesis at the ribosomal level (the site of protein synthesis), clindamycin is believed to kill bacteria associated with acne.

Benzoyl peroxide and clindamycin topical is a combination medicine used to treat acne.

There still is a widely recognized need for a composition comprising BPO and retinoid in which the active ingredients are chemically stable when formulated together in the same composition.

### SUMMARY OF THE INVENTION

The inventors of the present application have found that a composition comprising the active agent benzoyl peroxide (BPO) and adapalene, wherein at least one of the active agents are encapsulated in a microcapsule (coated by a shell), such a composition has significantly low irritation and high tolerability even when the active agents are present in amounts of at least 2% BPO and at least 0.10% adapalene (at least one encapsulated in a shell).

It is noted that Epiduo SBA indicated that compositions comprising 2.5% BPO and 0.1% adapalene have similar tolerability to compositions comprising 2.5% or 5% BPO. However, when accounting for the tolerability of compositions comprising 5% BPO and 0.1% adapalene this showed significantly more irritation that compositions going as high as 10% BPO.

Thus, the present invention provides a composition comprising as active agents:

benzoyl peroxide (BPO) in an amount of at least 2% wt and

adapalene in an amount at least 0.1% wt,

wherein at least one of the active agents is encapsulated in a microcapsule.

In various embodiments, the composition further comprises at least one additional agent. In some embodiments, the additional active agent is an antibiotic agent. In some embodiments, the antibiotic agent is clindamycin phosphate. In some embodiments, the clindamycin phosphate is in an amount of at 1.2% wt.

The present invention further provides a composition comprising as active agents:

benzoyl peroxide (BPO) in an amount of at least 2% wt;

adapalene in an amount of at least 0.1% wt; and

clindamycin phosphate in an amount of 1.2% wt,

wherein at least one of said active agents is encapsulated in microcapsules.

In some embodiments, the composition as described hereinabove comprises benzoyl peroxide (BPO) in an amount of 3% wt, and adapalene in an amount of 0.15%.

It is to be noted that each of the active agent may be either encapsulated in a microcapsule or non-encapsulated. Under embodiments when one or both active agents are encapsulated in a microcapsule, each of the active agent occupies the core of a single microcapsule.

In some embodiments, each of the active agents are encapsulated in microcapsules.

In other embodiments, the microcapsule has an inorganic shell. In other embodiments, the microcapsule has an inorganic oxide shell.

In further embodiments, the BPO is in the form of a solid particulate matter.

In some embodiments, the BPO is in an amount of at least 2.5% wt. In other embodiments the BPO is in an amount of at least 3% wt. In other embodiments the BPO is in an amount of at least 4% wt. In yet further embodiments, the BPO is in an amount of between 2.5%-5% wt.

In some embodiments, the adapalene is in an amount of at least 0.15% wt in the composition. In other embodiments, the adapalene is in an amount of at least 0.25% wt in the composition. In further embodiments, the adapalene is in an amount of between 0.2% wt to about 0.5% wt.

In some embodiments the microcapsule has a shell that is directly deposited on the active agent. In some embodiments, the shell is directly deposited on the BPO, forming a microcapsule comprising within its core BPO. In other embodiments, the shell is directly deposited on the adapalene, forming a microcapsule comprising within its core adapalene. In further embodiments, the shell is directly deposited on BPO and directly deposited on adapalene, forming two separate microcapsules each comprising within its core BPO and adapalene, respectively.

In other embodiments, the inorganic shell or inorganic oxide shell is directly deposited on the active agent. In some embodiments, the inorganic shell or inorganic oxide oxide shell is directly deposited on said BPO, forming a microcapsule comprising within its core BPO. In other embodiments, the inorganic shell or inorganic oxide shell is directly deposited on the adapalene, forming a microcapsule comprising within its core adapalene. In further embodiments, the inorganic shell or inorganic oxide shell is directly deposited on BPO and directly deposited on adapalene, forming two separate microcapsules each comprising within its core BPO and adapalene, respectively.

In some other embodiments, a composition of the invention has an improved stability as compared to a reference composition, the difference between the composition of the invention and the reference composition being in that in the reference composition the active ingredients are uncoated (unencapsulated, not encapsulated).

In some embodiments the composition of the invention further comprises at least one additional active agent. In some embodiments, the at least one additional active agent is at least one antibiotic agent. In some embodiments, the antibiotic agent is clindamycin phosphate. In some embodiments, the clindamycin phosphate is in an amount of at 1.2% wt.

According to this invention, “inorganic oxide” refers in one embodiment to metal oxide salt, in another embodiment to semi-metal oxide salt, and in another embodiment to a metalloid oxide salt. In other embodiments, inorganic oxide is selected from sodium silicate, potassium silicate, sodium aluminate, potassium aluminate, sodium titanate, potassium titanate, sodium zirconate, potassium zirconate, and mixtures thereof. In some other embodiments, the inorganic oxide salt is a silicate salt. In some embodiments, the inorganic oxide is selected from silica, titania, alumina, zirconia, ZnO, and mixtures thereof. In further embodiments, the inorganic oxide is silica.

In further embodiments, the composition of the invention is suitable for topical administration. In some further embodiments, the composition of the invention is in a gel form.

In some embodiments, the microcapsules are prepared by deposition of inorganic oxide on the surface of the solid particulate matter. In other embodiments, the microcapsules are prepared by oil-in-water in situ polymerization encapsulation processes.

In some embodiments, the microcapsule further comprises phase changing material (PCM) in the core. The preparation of microcapsules under this embodiment can be found in WO 2011/080741 which is herein incorporated by reference.

In some embodiments, the composition of the invention has reduced side effects as compared to a reference composition in which the active ingredients are uncoated. In some embodiments, the side effects are selected from at least one of irritation, erythema, stinging, itching, scaling, dryness, and combinations thereof.

The invention provides a method for treating a surface condition in a subject comprising topically administering onto the surface a composition of the invention. In some embodiments, the surface is skin or mucosal membrane. In other embodiments, the surface condition is selected from acne, rosacea, psoriasis, photoaging skin, hyperpigmented skin, mucosal infected areas, inflamed dermatitis, and combinations thereof.

The invention further provides a kit comprising:

(a) a first composition comprising BPO in the amount of at least 2% wt as a first active agent; and
(b) a second composition comprising adapalene in the amount of at least 0.1% wt as a second active agent:
at least one of the first and the second active agent being coated by a shell.

In some embodiments, the kit further comprises a third composition comprising an antibiotic. In some embodiments, the antibiotic is clindamycin phosphate in an amount of 1.2% wt.

In some embodiments, one of the first and the second active agent being coated by a shell and the other is present in an uncoated free form or in a coated form of the active ingredient. In some embodiments, the shell is an inorganic shell. In some embodiments, the shell is an inorganic oxide shell.

In other embodiments, the kit further comprises instructions for use in the treatment of a disease or disorder selected from one or more of acne, rosacea, psoriasis, photoaging skin, hyperpigmented skin, inflamed dermatitis, mucosal infected areas, the use comprising combining the first and the second composition for said treatment.

Moreover, the present invention relates to a method of using the kit as described in the present invention, wherein the first composition and the second composition are applied concomitantly or sequentially onto a surface of a subject's body.

The present invention relates to a composition for topical application comprising as active ingredients BPO and adapalene, wherein one of the active ingredients, i.e. BPO or adapalene, is in the form of first microcapsules comprising a solid particulate matter of the active ingredient coated by an inorganic shell or inorganic oxide shell and the other of the active ingredients is present in an uncoated free form or in a coated form of the active ingredient.

The present invention further relates to a composition for topical application as defined in the present invention the composition having reduced side effects as compared to a reference composition, in which the active ingredients are uncoated.

The present invention further relates to a method for treating a surface condition in a subject comprising topically administering onto the surface a composition as described in the present invention.

The present invention additionally relates to a method for preparing a composition comprising as active ingredients BPO and adapalene, which are chemically unstable when formulated together, and optionally a third active agent, wherein the composition exhibits improved stability of at least one of the active ingredients, the method comprising:

(a) separating the BPO and adapalene from each other in the composition by coating a solid particulate matter of one of the active ingredients by an inorganic oxide coating layer/shell to form first microcapsules, the other of the BPO and adapalene is incorporated into the composition in an uncoated free form or in a coated form of the active ingredient;
(b) optionally adding a third active ingredient to the composition; and
(c) adding excipients for the preparation of the composition.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows the sum of severance of erythema score from all sites on animals conducted by two reviewers during drug product (DP) dosing and follow-up period for DPs according to the present invention, EpiDuo® Gel and untreated group.

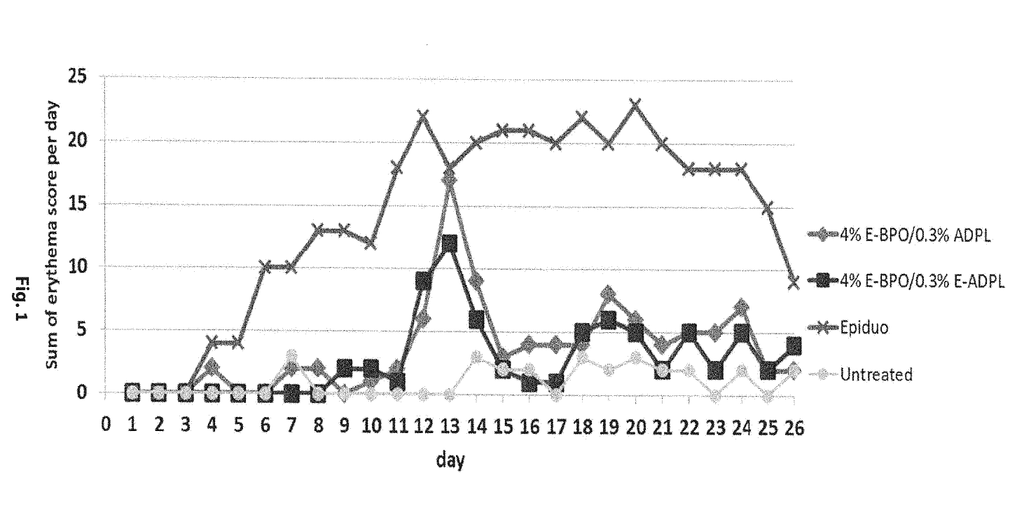

### DETAILED DESCRIPTION OF THE INVENTION

The present invention is based on the findings that it is possible to formulate two or more reactive active agents in the same composition. Surprisingly it was found in the present invention that it is possible to formulate a peroxide (particularly benzoyl peroxide) and a retinoid (particularly adapalene) which are chemically reactive, in the same composition by coating a solid particulate matter of one of these active agents (or each of these active agents) for example by an inorganic coating/inorganic oxide coating, thus separating these two active agents from each other in the same composition. Such a composition was found to be advantageous with respect to the chemical stability of the active ingredients and further found to have reduced side effects as compared to a reference composition comprising the uncoated active agents.

Thus, the present invention relates to a composition for topical application comprising as active ingredients BPO and adapalene, wherein one of the active ingredients (BPO or adapalene) is in the form of first microcapsules comprising a solid particulate matter of the active ingredient coated by an inorganic shell or inorganic oxide shell and the other of the active ingredients is present in an uncoated free form or in a coated form of the active ingredient. The composition may further comprise a third active agent, e.g., clindamycin phosphate, which is present either in an uncoated free form or in a coated form as described hereinabove.

As used herein unless otherwise indicated the term “microcapsule” refers to a microparticle having a core shell structure, wherein said core comprises an active agent as defined herein (core material), being coated by a shell forming the microcapsule entrapping the core. In some embodiments, the coating/shell is directly deposited on the core material. In some embodiments, the core material is solid. In other embodiments, the core material is semi-solid. In some embodiments, the core material consists of a solid particle of the active agent. In other embodiments, the core material comprises a solid particle of the active agent. In some other embodiments, the core material is in a liquid/oily phase.

The size of the microcapsules (denoted herein also by the general term “particles” or “microparticles”) as will be referred to herein refers to D₉₀ meaning that 90% of the particles have the stated dimension or less (measured by volume). Thus, for examples, for spherical particles stated to have a diameter of 10 micrometers (“microns”), this means that the particles have a D₉₀ of 10 microns. The D₉₀ (termed also d(0.9)) may be measured by laser diffraction. For particles having a shape other than spheres, the D₉₀ refers to the mean average of the diameter of a plurality of particles.

Sol-Gel process has been used to encapsulate various active ingredients, thus isolating the active ingredient from the environments. In some embodiments, the microcapsules are formed using a sol-gel process as disclosed in the following documents (herein incorporated by reference): U.S. Pat. Nos. 6,303,149, 6,238,650, 6,468,509, 6,436,375, US2005037087. US2002064541, and International publication Nos. WO 00/09652, WO00/72806, WO 01/80823, WO 03/03497. WO 03/039510, WO00/71084, WO05/009604, and WO04/81222, disclose sol-gel microcapsules and methods for their preparation; EP 0 934 773 and U.S. Pat. No. 6,337,089 teach microcapsules containing core material and a capsule wall made of organopolysiloxane, and their production; EP0941 761 and U.S. Pat. No. 6,251,313 also teach the preparation of microcapsules having shell walls of organopolysiloxane; U.S. Pat. No. 4,931,362 describes a method of forming microcapsules or micromatrix bodies having an interior water-immiscible liquid phase containing an active, water-immiscible ingredient. Microcapsules prepared by a sol-gel process are also disclosed in GB2416524, U.S. Pat. No. 6,855,335, WO03/066209.

According to some embodiments of the present invention, the coated form of the active ingredient (microcapsule) may be in form of a polymeric microsponge where the active ingredient is adsorbed or entrapped in the microsponge as described for example in U.S. Pat. Nos. 4,690,825; 5,145,675, 5,879,716 and 5,955,109, incorporated herein by reference in their entirety.

In other embodiments, microcapsules are formed by the encapsulation process disclosed in the following publications (herein incorporated by reference): U.S. Pat. No. 7,629,394, U.S. Pat. No. 9,205,395, US 2015/0328615, US 2014/0186630. Controlled release microcapsules: IN01958CH2007, IN02080CH2007, U.S. Pat. No. 4,235,872, U.S. Pat. No. 4,670,250, EP 0248531, U.S. Pat. No. 4,970,031, U.S. Pat. No. 5,238,714, WO9321764, U.S. Pat. No. 5,575,987, WO9420075, US 2004/137031, US 2006/003014, US 2010/180464.

The core (wherein it is a solid particulate matter) may be of any shape for example rod-like, plate-like, ellipsoidal, cubic, or spherical shape.

In the case of cores having a spherical shape, the diameter (D₉₀) may be in the range of 0.3 to 90 microns, in some embodiments 0.3 to 50 microns, in some further embodiments 1 to 50, in some further embodiments 5 to 30 microns.

By the term “the diameter (D90) may be in the range of 0.3 to 90 microns” is meant that 90% by volume of the particles (in this case the particle's core) may be less than or equal to a value in the range of 0.3 to 90 microns.

For generally cubic-shaped cores or cores having a shape resembling that of a cube, the mean size of a side may be in the range 0.3 to 80 microns, in some embodiments 0.3 to 40 microns, in some further embodiments 0.8 to 40 microns, in some further embodiments 4 to 15 microns.

For rod-like shaped, ellipsoidal-shaped and plate-like shaped cores, the largest dimension (that of the longest axis) is typically in the range 10 to 100 microns, in some embodiments 15 to 50 microns; and the smallest dimension is typically in the range 0.5 to 20 microns and in some further embodiments 2 to 10 microns.

According to an embodiment of the present invention, the microcapsules (coated particulate matter) have a diameter (d₉₀) of 0.5 to 100 μm or in some embodiments the diameter of the microcapsules is in the range of 1 to 50 μm and in some other embodiments in the range of 5 to 30 μm. It is appreciated that the microcapsules of the present invention are composed of distinct regions of the inorganic shell or inorganic oxide shell in the core material (i.e. the water insoluble particulate matter).

Further according to an embodiment of the present invention the obtained inorganic oxide coating shell has a width (thickness) of 0.1 μm or above, in some embodiments the inorganic oxide coating shell has a width (thickness) of 0.1-10 μm.

Additionally, according to an embodiment of the present invention the obtained inorganic oxide coating shell has a width (thickness) of 0.3 μm or above, in some embodiments the inorganic oxide coating shell has a width of 0.3-10 μm.

Additionally, according to an embodiment of the present invention, the thickness of the inorganic oxide shell is in the range of 0.1-10 μm. In some further embodiments, the thickness of the inorganic oxide shell is in the range of 0.1-3 μm, and in some further embodiments in the range of 0.1-1 μm. The thickness of the inorganic oxide shell may also be in some embodiments in the range of 0.3 to 3 μm, and in some other embodiments in the range of 0.3 to 2 μm.

Further according to an embodiment of the present invention the obtained inorganic oxide coating shell has a width (thickness) of about 0.1, 0.2, 0.3, 0.5, 0.7, 1, 1.5, 2 or 5 μm or above, in some embodiments up to 10 μm.

The width of the inorganic shell or inorganic oxide shell may be determined for example by a Transmission Electron Microscope or Confocal Microscope such that in a circular cross sectional area of the particle the smallest width is at least e.g. 0.1 μm (the width is determined as the smallest distance from the surface of the particle (i.e. inorganic oxide surface) to the core-inorganic oxide interface).

The microcapsules are in some embodiments characterized in that the core material is substantially free of the inorganic oxide and further in that the inorganic oxide shell is substantially free of the core material, e.g. either as particle dispersion (in the nano-metric range of below 0.1 μm) of the particulate matter or as molecular dispersion of the particulate matter.

Thus, according to an embodiment of the present invention, the inorganic shell or inorganic oxide shell is substantially free of core material (either in the form of molecules or as nano-metric particles). The term “substantially fee” in this context denotes that the concentration of the molecules of the core material or the concentration of the nano-metric particles of the core material is negligible as compared to the inorganic oxide. Similarly, by the term “the core material is substantially free of the inorganic oxide” is meant that the concentration of the inorganic oxide in the core is negligible as compared to the core material. The microcapsules (i.e. first microcapsules) are in some embodiments non leaching when dispersed in a carrier and in some other embodiments non leaching in an aqueous based carrier.

According to another embodiment when the microcapsules are prepared by a method such as spray drying, the core material comprising the active agent may further comprise up to about 30% w/w, in some embodiments up to about 20% inorganic oxide and the inorganic oxide coating layer may further comprise up to about 30% w/w, in some embodiments up to about 20% w/w of the active agent.

By the term “non-leaching” it is meant that the leaching of the particulate matter (active agent) from the particles into an aqueous-based liquid is less than 5% w/w, in some embodiments less than 3%, in some further embodiments less than 1% w/w in some further embodiments less than 0.5% w/w, and in some other embodiments less than 0.1% w/w at room temperature (20° C.), under gentle agitation for 1 hour or until a steady state concentration is achieved. Typically, the aqueous-based liquid is water. The values indicated above refer to the percentage of the active agent leached into an aqueous medium relative to the initial amount of the active agent in the particles. The leaching values indicated above refer in some embodiments to a dispersion having a concentration of the particulate matter in the aqueous medium higher than 0.1% w/w, in some further embodiments higher than 1% w/w, in some further embodiments higher than 3% w/w, and in some other embodiments higher than 10% w/w. For adapalene the leaching values indicated above refer in some embodiments to a dispersion having a concentration of the particulate matter in the aqueous medium higher than 0.01% w/w.

According to an embodiment of the present invention the weight ratio of the inorganic oxide to the solid particulate matter is in the range of 1:99 to 50:50. The weight ratio of the inorganic oxide shell to the solid particulate matter may be also in the range of 3:97 to 50:50, 5:95 to 50:50, 10:90 to 50:50, 5:95 to 30:70, 10:90 to 30:70. Further, according to an embodiment of the present invention the rate ratio of the inorganic oxide to the solid particulate matter is in the range of 10:90 to 20:80.

According to another embodiment of the present invention, when spray drying method is used, the weight ratio of the inorganic oxide to the solid particulate matter may be in the range 5:95 to 95:5.

As used herein by the term “uncoated free form” is meant that the active ingredient (e.g., BPO, adapalene, clindamycin phosphate) is present in the composition in its “naked” form meaning that it is not intimately embedded, encapsulated, entrapped or encased in a polymeric carrier, and is present in the composition in direct contact with the composition carrier.

As used herein by the term “coated form of the active ingredient” is meant that the active ingredient is embedded, dispersed, entrapped, or encased, e.g. as a solid dispersion or molecular dispersion in a polymeric carrier which may be an organic or inorganic carrier and which may serve as a matrix for dispersing the active ingredient or as encapsulated material coating said active ingredient (i.e the active ingredient is present in a core or is a core material encapsulated by a shell composed of a polymeric material which may be an organic or inorganic polymer).

According to a more preferred embodiment of the present invention, the coated form of the active ingredient is second microcapsules comprising a solid particulate matter of the active ingredient coated by an inorganic shell or an inorganic oxide shell.

Further, according to an embodiment of the present invention, the first microcapsules comprise a solid particulate matter of BPO coated by an inorganic oxide shell.

According to an embodiment of the present invention, the BPO is in the form of first microcapsules comprising solid particulate matter of BPO coated by an inorganic shell or an inorganic oxide shell and the adapalene is in the form of second microcapsules comprising a solid particulate matter of the adapalene coated by an inorganic shell or an inorganic oxide shell.

Under these embodiments, the inorganic coating shell or inorganic oxide coating shell is advantageous since it is capable of isolating the particulate matter of the active agent from its surrounding medium, thus preventing cross-reactivity of the active agents present in the same composition and yet enables the release the particulate matter upon application to the surface to be treated.

The term “solid water insoluble agent” refers to a solid material having solubility in water of less than 3% w/w, typically less than 1% and at times less than 0.5% w/w at room temperature (20° C.). The “solid water insoluble agent” may have a solubility of less than 0.1% w/w.

The “solid water insoluble agent” may also be termed herein as “solid water insoluble particulate matter” or “solid particulate matter”.

The composition of the present invention comprises a carrier. According to an embodiment of the present invention the carrier is in the form of an ointment, a cream, a lotion, an oil, a solution (in some embodiments an aqueous solution), an emulsion, a gel, a paste, a milk, an aerosol, a powder, or a foam. In some embodiments the carrier is an aqueous-based carrier (such as a gel, oil-in water emulsion or oil-in water cream, aqueous solution, foam, lotion, spray).

Thus, the final form of the composition may be any of the above forms, mentioned with respect to the carrier, where the microcapsules are dispersed in the carrier. The final form of the composition may also be in the form of a wash or cleanser.

Moreover, according to an embodiment of the present invention, the composition having an improved stability as compared to a reference composition the difference between the composition of the present invention and the reference composition being in that the reference composition and the active ingredients are uncoated.

As used herein by the term “improved stability” is meant that the degradation of the adapalene in the presence of the peroxide (e.g. benzoyl peroxide) is in some embodiments less than 30%, in some further embodiments less than 20%, in some further embodiments less than 10% of the initial retinoid concentration in a time range of 3 months at room temperature (20-25° C.), or 1 month at 30° C.

According to an embodiment of the present invention the composition further comprising an additional active agent.

In some embodiments the additional active agent is an antibiotic agent. In some further embodiments the antibiotic agent is an antibiotic of the lincomycin family. In some other embodiments the antibiotic of the lincomycin family is clindamycin, a pharmaceutical acceptable salt thereof, or an ester thereof. In some other embodiments the antibiotic agent is clindamycin phosphate.

The antibiotic may be present in an uncoated free form or in a coated form of the active ingredient. The uncoated free form and coated free form may be as described in the present invention with respect to the BPO and adapalene.

In various embodiments, the additional active ingredient is in the form of third microcapsules comprising a solid particulate matter of the ingredient coated by an inorganic shell or an inorganic oxide shell. In some embodiments, the active ingredient is clindamycin phosphate.

Therefore, according to some embodiments of the present invention, the BPO is in the form of first microcapsules comprising solid particulate matter of BPO coated by an inorganic shell or an inorganic oxide shell, the adapalene is in the form of second microcapsules comprising a solid particulate matter of the adapalene coated by an inorganic shell or an inorganic oxide shell, and the antibiotic (e.g., clindamycin phosphate) is in the uncoated free form.

According to another embodiment of the present invention, the BPO is in the form of first microcapsules comprising solid particulate matter of BPO coated by an inorganic shell or an inorganic oxide shell, the adapalene is in the form of second microcapsules comprising a solid particulate matter of the adapalene coated by an inorganic shell or an inorganic oxide shell and the antibiotic (e.g., clindamycin phosphate) is in the form of third microcapsules comprising a solid particulate matter of the antibiotic coated by an inorganic shell or an inorganic oxide shell.

In some embodiments, the inorganic oxide is selected from silica, titania, alumina, zirconia, ZnO, and mixtures thereof. In some other embodiments the inorganic oxide is silica.

Moreover, according to an embodiment of the present invention, the microcapsules (coated particulate matter) have a diameter of 0.5-100 μm. In some embodiments, the particles have a diameter of 0.8-100 μm, in some further embodiments 1-50 μm and in some other embodiments 2-30 μm.

According to certain embodiments of the present invention, the surface of the inorganic shell or inorganic oxide shell of the coated particulate matter may be chemically modified by organic groups, in some embodiments hydrophobic groups, attached to its surface.

The hydrophobic groups may be for example an alkyl groups (such alkyl groups may be further substituted with one or more fluoro atoms), aryl groups (such as benzyl or phenyl), and combinations thereof. The groups may be as described below with respect to the process.

Further according to an embodiment of the present invention, the first microcapsules are prepared by deposition of inorganic oxide on the surface of the solid particulate matter. The deposition of inorganic oxide on the surface of the particulate matter may be performed by precipitation of an inorganic oxide salt onto the surface of the particulate matter, forming an inorganic oxide shell thereon as will be described below or by a spray drying method.

In some embodiments the first microcapsules are prepared by a process comprising:

(a) contacting the solid, water-insoluble particulate matter, with an ionic additive and an aqueous medium to obtain a dispersion of the particulate matter having positive charges on its surface:
(b) coating the solid, water-insoluble particulate matter, by precipitation of an inorganic oxide salt onto the surface of the particulate matter, forming an inorganic oxide coating shell thereon; and
(c) aging said coating layer.

Still further according to an embodiment of the present invention, the first microcapsules are prepared by a process for coating a solid, water-insoluble particulate matter, with an inorganic oxide comprising:

(a) contacting the solid, water-insoluble particulate matter with an ionic additive and an aqueous medium to obtain a dispersion of the particulate matter having positive charges on its surface;
(b) subjecting the particulate matter to a coating procedure comprising precipitating an inorganic oxide salt onto the surface of the particulate matter to form an inorganic oxide shell thereon thereby to obtain particulate matter coated by an inorganic oxide coating shell;
(c) repeating step (b) at least 4 more times; and
(d) aging said coating layer.
In the process described the solid, water-insoluble particulate matter refers to the BPO or adapalene. The process described may also be used to coat additional active ingredients (e.g. antibiotics, e.g., clindamycin phosphate) which may be incorporated into the composition described in the present invention.

Step (a) of the process may further comprise reducing the particle size of the particulate matter to the desired particle size for example by milling, or homogenization.

In some embodiments step (c) of the process described above is repeated 4 to about 1000 times. This means that in some embodiments step (b) of the process described above is repeated 4 to about 1000 times.

In some embodiments the process comprising repeating step (c) 4 to about 300 times, and in some further embodiments 4 to about 100 times. In some further embodiments step (c) of the process described above is repeated 5-80 times and in some other embodiments 5-50 times. This means that in some embodiments step (b) is repeated as indicated above with respect to step (c).

According to an embodiment of the present invention the process comprising:
(a) contacting the solid, water-insoluble particulate matter, with a first cationic additive and an aqueous medium to obtain a dispersion of the particulate matter having positive charges on its surface;
(b) subjecting the particulate matter to a coating procedure comprising precipitating an inorganic oxide salt onto the surface of the particulate matter to form an inorganic oxide coating shell on the particulate matter;
(b1) in an aqueous medium contacting the coated particulate matter with a surface adhering additive being one or both of (i) a second cationic additive, and (ii) a non-ionic additive;
(b2) subjecting the particulate matter obtained in step (b1) to a coating procedure as in step (b);
(c) repeating steps (b1) and (b2) at least 3 more times; and
(d) aging the inorganic oxide coating shell.

Additionally, according to an embodiment of the present invention the process comprising:
(a) contacting the solid, water-insoluble particulate matter, with an anionic additive, a first cationic additive and an aqueous medium to obtain a dispersion of the particulate matter having positive charges on its surface;
(b) subjecting the particulate matter to a coating procedure comprising precipitating an inorganic oxide salt onto the surface of the particulate matter to form an inorganic oxide coating shell on the particulate matter;
(b1) in an aqueous medium contacting the coated particulate matter with one or both of (i) a second cationic additive, and (ii) a non-ionic additive;
(b2) subjecting the particulate matter obtained in step (b1) to a coating procedure as in step (b);
(c) repeating steps (b1) and (b2) at least 3 more times; and
(d) aging the inorganic oxide coating shell.

According to the present invention, the inorganic oxide refers in one embodiment to metal oxide salt and in another embodiment to a metalloide oxide salt, and is selected from sodium silicate, potassium silicate, sodium aluminate, potassium aluminate, sodium titanate, potassium titanate, sodium zirconate, potassium zirconate, and mixtures thereof. In some other embodiments, the inorganic oxide salt is a silicate salt.

According to certain embodiments, the process may further comprise adding a colloidal inorganic oxide suspension, in some embodiments aqueous-based suspension (comprising nanometric inorganic oxide (nanoparticles of inorganic oxide)) during the coating procedure. In some embodiments the colloidal inorganic oxide suspension is selected from colloidal silica suspension, colloidal titania suspension, colloidal alumina suspension, colloidal zirconia suspension, colloidal ZnO suspension, and mixtures thereof. The colloidal inorganic oxide suspension may be added during the coating process (e.g. in step (b) in one or more of its repeated steps). In some embodiments the size of the nanometric inorganic oxide in diameter is in the range between 5-100 nm (average particle size diameter). The weight ratio of the nanometric inorganic oxide to the inorganic oxide salt may be in the range of 95:5 to 1:99, in some embodiments in the range of 80:20 to 5:95, in some further embodiments in the range of 70:30 to 10:90, in some other embodiments about 60:40 to 20:80. The weight ratio of the nanometric inorganic oxide to the inorganic oxide salt may be about 50:50.

According to certain embodiments, the particles (first and/or second microcapsules of the invention) may be characterized in that when tested in a Dissolution Tester using Paddle Method in a medium, typically an organic-based solvent such as acetonitrile, isopropyl myristate, ethanol or methanol, in which the particulate matter is soluble, and a dissolution volume in which the concentration of the particulate matter is lower than the solubility of the particulate matter, the time for releasing 50% w/w of the particulate matter from the particles is at least two-fold higher, in some embodiments at least three-fold higher, in some embodiments at least four-fold higher, in some further embodiments at least five-fold higher and in some other embodiments at least ten-fold higher as compared to the dissolution of the free form of the particulate matter having substantially the same particle size diameter as the particulate matter in said particles.

The dissolution of the free form of the particulate matter is measured under the same conditions as the coated particulate matter. The time for releasing 50% w/w of the particulate matter (active agent) from the particles is compared to the time of 50% w/w dissolution of the free form. In some embodiments the dissolution volume is such that the concentration of the particulate matter is lower than at least half of the solubility of the particulate matter. The “solubility” relates to the solubility of the particulate matter (active ingredient) in the dissolution medium (e.g. an organic-based solvent such as acetonitrile, isopropyl myristate, ethanol or methanol). It is appreciated that the dissolution volume will also depend on the detection level of the analytical method. The dissolution may be conducted at a temperature of 20° C.-40° C. The dissolution may be conducted at a paddle rate of 50-200 rpm.

According to a specific embodiment the dissolution of the particles is as described above, when the particles are prepared by the repetitive coatings steps as described in the process above.

According to a specific embodiment the particles are prepared by the repetitive coating steps as described in the process above.

The first microcapsules may also be prepared by a process as disclosed in co-owned PCT application, publication number WO 2007/015243, the content of which is incorporated herein by reference.

In some embodiments, the microcapsule further comprises phase changing material (PCM) in the core. The preparation of microcapsules under this embodiment can be found in WO 2011/080741, which is herein incorporated by reference.

Thus, in some embodiments the process for preparing microcapsules having a core encapsulated within an inorganic shell or inorganic oxide shell, the process comprising:
a) preparing an oil-in-water emulsion by emulsification of an oily phase comprising at least one active agent and at least one phase changing material, in an aqueous phase, wherein at least one of the oily phase and aqueous phase comprise a sol-gel precursor;
b) subjecting the emulsion to microcapsule forming conditions; thereby obtaining said microcapsules.

Thus, the core of a microcapsule under these embodiments refers to the inside part of the microcapsules comprising at least one active agent and at least one phase changing material that are both surrounded by an inorganic shell or inorganic oxide shell of a microcapsule. It should be noted that additional compounds may be present in the core including for example carriers, excipients, pharmaceutically acceptable polymers or salts etc, all in accordance with the intended use of produced microcapsules, which will be apparent to a skilled artisan preparing said microcapsules. The core of said microcapsule of the invention may comprise said at least one active agent and at least one phase forming material.

In some embodiments the viscosity of the core at room temperature may be about 300 cP, 350 cP, 400 cP, 450 cP, 500 cP, 550 cP, 600 cP, 650 cP, 700 cP, 750 cP, 800 cP, 900 cP, 1000 cP, 2000 cP, 3000 cP, 4000 cP, 5000 cP, 6000 cP, 7000 cP, 8000 cP, 9000 cP, 10,000 cP, 20,000 cP, 30,000 cP, 40,000 cP, 50,000 cP, 60,000 cP, 70,000 cP, 80,000 cP, 90,000 cP, 100,000 cP, 200,000 cP, 300,000 cP, 400,000 cP, 500,000 cP, 600,000 cP, 700,000 cP, 800,000 cP, 900,000 cP or 1,000,000 cP (when measured under various conditions). In some embodiments the viscosity of the core at room temperature is between about 300 to 600 cP. In other embodiments the viscosity of the core at room temperature is between about 400 to 500 cP. In further embodiments the viscosity of the core at room temperature is between about 300 to 10,000 cP. In other embodiments the viscosity of the core at room temperature is between about 5.000 to 1,000,000 cP. In some further embodiments the viscosity of the core at room temperature is between about 20,000 to 1,000,000 cP.

In other embodiments of the invention the core may be solid at room temperature. In other embodiments, the core may be in a semi-solid phase at room temperature.

The oily phase utilized by a process of the invention comprises at least one active agent and at least one phase changing material. The at least one active agent may be in a form of a water insoluble liquid or dispersion in water-insoluble liquid comprising the at least one active agent.

The term “phase changing material” (PCM) is meant to encompass any substance capable of changing its state of matter (phase), or at least its viscosity, in accordance with the temperature it is exposed to. PCMs typically have a high heat of fusion which enables them to melt and solidify at certain temperatures and are capable of storing and releasing large amounts of energy. Heat is absorbed or released when the PCM material changes from solid to liquid and vice versa. When PCMs reach the temperature at which they change phase or viscosity (for example their melting temperature), they absorb large amounts of heat at an almost constant temperature. The PCM continues to absorb heat without a significant raise in temperature until all the material is transformed to the liquid phase. When the ambient temperature around a liquid material falls, the PCM solidifies, releasing its stored latent heat. In accordance with an embodiment of the present invention a phase changing material utilized by a process of the invention is an organic material, which is non-reactive with any compound utilized by a process of the invention and is characterized by the fact that at room temperature said PCM has a viscosity of between about 300 cP to 1,000,000 cP (when measured under various conditions). In some embodiments the viscosity of said PCM at room temperature may be 300 cP, 350 cP, 400 cP, 450 cP, 500 cP, 550 cP, 600 cP, 650 cP, 700 cP, 750 cP, 800 cP, 900 cP, 1000 cP, 2000 cP, 3000 cP, 4000 cP, 5000 cP, 6000 cP, 7000 cP, 8000 cP, 9000 cP, 10,000 cP, 20,000 cP, 30,000 cP, 40,000 cP, 50.000 cP, 60,000 cP, 70,000 cP, 80.000 cP, 90,000 cP, 100,000 cP, 200.000 cP, 300,000 cP, 400,000 cP, 500,000 cP, 600,000 cP, 700,000 cP, 800,000 cP, 900,000 cP or 1,000.00 cP (when measured under various conditions).

In one embodiment, the at least one phase changing material is selected from natural or synthetic paraffins (e.g. having a molecular formula of CₙH₂ₙ₊₂, wherein n=10-100), C₁₀-C₁₀₀ alkane, C₁₀-C₁₀₀ alkene (having at least one double bond), C₁₀-C₁₀₀ alkyne (having at least one triple bond), aliphatic alcohols (e.g. having a molecular formula of CH₃(CH₂)ₙOH, wherein n=10-100) and fatty acids (e.g. having a molecular formula of CH₃(CH₂)₂ₙCOOH, wherein n=10-100), or any combination thereof.

In some embodiments, the at least one phase changing material is at least one natural or synthetic paraffin. In some embodiments, the at least one phase changing material is a C₁₀-C₁₀₀ aliphatic alcohol (in other embodiments C₁₀, C₂₀, C₃₀, C₄₀, C₅₀, C₆₀, C₇₀, C₈₀, C₉₀ to C₁₀₀ aliphatic alcohol). In further embodiments, the at least one phase changing material is a C₁₀-C₁₀₀ aliphatic fatty acid (in other embodiments C₁₀, C₂₀, C₃₀, C₄₀, C₅₀, C₆₀, C₇₀, C₈₀, C₉₀ to C₁₀₀ aliphatic fatty acid).

The BPO and adapalene combination, and optionally clindamycin phosphate, in the same composition, can be designed to have differentiation in the release profile of the active agents by modification of the inorganic oxide (e.g. silica) shell formed on each active agent. Microcapsules comprising BPO for example can have a thick silica layer thus providing a slow release profile while microcapsules comprising adapalene and/or clindamycin phosphate can have a thin silica layer or the adapalene and/or clindamycin phosphate can have no coating layer at all, thus providing a fast release profile.

The invention additionally relates to a composition for topical application as defined in the present invention the composition having reduced side effects as compared to a reference composition in which the active ingredients are uncoated.

According to an embodiment of the present invention the side effects are selected from irritation, erythema, stringing, itching, scaling, dryness, and combinations thereof. The side effects may also be other similar dermal undesirable side effect.

The invention further relates to a method for treating a surface condition in a subject comprising topically administering onto the surface a composition as described in the present invention

In some embodiments the surface is skin or mucosal membrane. In some embodiments the surface condition is selected from acne, rosacea, psoriasis, photoaging skin, hyperpigmented skin, mucosal infected areas, inflamed dermatitis, and combinations thereof.

Such surface conditions are in some embodiments treatable by retinoids and peroxides.

In some embodiments the subject is a mammal and in some other embodiments the mammal is a human.

The term “treating or treatment” as used herein includes any treatment of a condition (disease or disorder such as acne, rosacea, psoriasis, and combinations thereof) associated with a patient's body surface such as the skin or mucosal membrane and includes inhibiting the disease or disorder (i.e. arresting its development), relieving the disease or disorder (i.e. causing regression of the disease or disorder) or relieving the conditions caused by the disease (i.e. symptoms of the disease).

The concentrations of the active ingredients that can be used for treatment of a specific disease or disorder may be 2%-20% w/w BPO and 0.2%-0.5% w/w adapalene, in some embodiments 2.5%-15% w/w BPO and 0.3%-0.4% w/w adapalene, in some other embodiments 2.5%-10% w/w BPO and 0.3%-0.5% w/w adapalene, in some other embodiments 2.5%-10% w/w BPO and 0.1%-0.5% w/w adapalene, in some other embodiments 2.5%-10% w/w BPO and 0.1%-0.2% w/w adapalene, based on the total weight of the composition.

In various embodiments, the concentration of the additional active ingredient (e.g., clindamycin phosphate) that can be added to the composition, may be 0.1%-20% w/w, in some embodiments 0.5%-10% w/w, in some other embodiments 1%-5% w/w, in some embodiments 1.2% w/w, based on the total weight of the composition. Although individual needs may vary, determination of optimal ranges for effective amounts of the composition is within the skill of the art. Generally, the dosage required to provide an effective amount of a composition which can be adjusted by one skilled in the art will vary depending on the age, health, physical condition, weight, type and extent of the disease or disorder of the recipient, frequency of treatment, the nature of concurrent therapy, if any, and the nature and scope of the desired effect.

Moreover, the present invention relates to a method for preparing a composition comprising as active ingredients BPO and adapalene, which are chemically unstable when formulated together, wherein the composition exhibits improved stability of at least one of the active ingredients, the method comprising:
(a) separating the BPO and adapalene from each other in the composition by coating a solid particulate matter of one of the active ingredients by an inorganic oxide coating shell to form first microcapsules, the other of the active ingredients is incorporated into the composition in an uncoated free form or in a coated form of the active ingredient; and
(b) adding excipients for the preparation of the composition.

In some embodiments, the composition may further comprise an additional agent, preferably antibiotics, more preferably, Clindamycin Phosphate.

Accordingly, the present invention further relates to a method for preparing a composition comprising as active ingredients BPO and adapalene, which are chemically unstable when formulated together, and a third ingredient (e.g. Clindamycin Phosphate), wherein the composition exhibits improved stability of at least one of the active ingredients, the method comprising:
(a) separating the BPO and adapalene from each other in the composition by coating a solid particulate matter of one of the active ingredients by an inorganic oxide coating shell to form first microcapsules, the other of the active ingredients is incorporated into the composition in an uncoated free form or in a coated form of the active ingredient;
(b) adding a third active ingredient to the composition; and
(c) adding excipients for the preparation of the composition.

As used herein the term “chemical unstable” refers to active ingredients which degrade, decompose, and/or chemically react one with the other resulting in a decrease of the active ingredient initial concentration. The term “chemical unstable” encompasses also “photochemical instability” as a result of light irradiation. In some embodiments the improved stability refers to the retinoid.

As used herein by the term “separating” is meant that above 90% w/w, in some embodiments above 95% w/w and in some further embodiments above 99% w/w of the total initial amount of the BPO present in the composition and above 90% w/w, in some embodiments above 95% w/w and in some further embodiments above 99% w/w of the total initial amount of the adapalene present in the composition are separated (i.e. not in direct contact or not intimately mixed) from each other in the same composition.

In some embodiments, the coated form of the active ingredient is prepared by coating a solid particulate matter of the active ingredient by an inorganic coating shell or an inorganic oxide coating shell to form second microcapsules. In some embodiments the coating is as described in the present invention.

The present invention further relates to a kit comprising: (a) a first composition comprising BPO as a first active ingredient; and (b) a second composition comprising adapalene as a second active ingredient; at least one of the first and the second active ingredient being coated by an inorganic shell or an inorganic oxide shell.

In some embodiments one of the first and the second active ingredient being coated by an inorganic shell or an inorganic oxide shell and the other is present in an uncoated free form or in a coated form of the active ingredient.

According to an embodiment the kit further comprises a third composition comprising a third active ingredient, which may be either coated by an inorganic shell or an inorganic oxide shell or in the uncoated free form. In some embodiments, the third active ingredient is an antibiotic. In some embodiments, the third active ingredient is Clindamycin Phosphate.

According to an embodiment the kit further comprises instructions for use in the treatment of a disease or disorder selected from one or more of acne, rosacea, psoriasis, photoaging skin, hyperpigmented skin, inflamed dermatitis, mucosal infected areas, the use comprising combining said first and said second composition for said treatment.

The present invention additionally relates to a method of using the kit as described in the present invention, wherein the first composition, the second composition and optionally the third composition, are applied concomitantly or sequentially (one subsequent to the other) onto a surface of a subject's body.

When referring to pharmaceutical compositions comprising a compound of the subject invention it should be understood to encompass admixtures of microcapsules of the invention, with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intrathecal) administration or administration via an implant. The compositions may be prepared by any method well known in the art of pharmacy. Such methods include the step of bringing in association compounds used in the invention or combinations thereof with any auxiliary agent.

Auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as cream, paste, gel, solution, emulsion or suspension.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injections. The compositions may be presented in unit-dose or multi-dose containers, for example sealed vials and ampoules, and may be stored in a freeze-dried (lyophilized) condition requiring only the addition of sterile liquid carrier, for example water, prior to use.

For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

### EXAMPLES

In the examples below, all % values referring to a solution are in (w/w).

All % values, referring to dispersions (suspensions) are in (w/w).
Unless otherwise indicated, all solutions used in the example below refer to an aqueous solution of the indicated ingredient.

### Example #1: Silica Encapsulation (Coating) of BPO

Step 1: Milling:

110 g. of hydrous BPO 75% (USP grade from Sigma. USA) were suspended in 152 g. of 0.4% CTAC solution containing 0.001% silicon antifoam. The BPO was milled using a stator rotor mixer (IKA 6100 operated at 15,000 rpm). The milling was stopped when the particle size distribution (PSD) of the suspension was d(0.9)≤35 μm or the temperature has reached 50° C. The final suspension was cooled to room temperature.

Step 2: Coating:

During the coating procedure the suspension was stirred with a mechanical dissolver, 60 mm, at 500 RPM at all times. The pH of the milled BPO suspension was corrected to 8 using NaOH 5N solution. A portion of 1 g of 15% sodium silicate solution (15% w/w as SiO₂) was added and the suspension was stirred for 5 min. A portion of 1 g of 3% Polyquatemium 7 (Poly diallyl ammonium chloride) was added and the suspension was stirred for 5 min. pH was adjusted to 6-7 using 5N HCl solution.

This procedure was repeated for 5-100 times in order to create a series of silica layers around BPO having different thicknesses.

### The Aging Step:

The coated BPO suspension at pH 6.5 was kept for aging at room temperature under gentle agitation for 24 hrs.

### Example #2: Analytical Evaluation of the BPO Release

The release profile of BPO out of the silica shell was conducted in a water/Acetonitrile solution, which is capable of dissolving BPO. The method is based on the strong oxidation properties of BPO. BPO reacts with I⁻ ions to form I₂, which gives a color reaction. I₂ is then reduced back to I⁻ using sodium thiosulfate (STS) to eliminate the color. Each 12.11 mg of oxidizing BPO can be reduced by 1 ml of 0.1M STS. The evaluation of BPO release was conducted using Solution A and Suspension B as detailed below.

Composition of 100 g. solution A, (capable to distinguish release of 30% BPO): 55 g. Acetonitrile, 12.4 g. 0.1M STS, 4.5 g. KI, 28.1 g. deionized water.

Suspension B, preparation of BPO: weigh 200 mg of BPO as 100% (1 g as 20% BPO suspension into 5 ml measuring bottle and fill with deionized water up to 5 ml.

Procedure: Into 50 ml glass beaker add 40 ml of solution A and the 5 ml of suspension B. Measure the time for yellow color appearance.

TABLE 1

| | Number of | Time for color |
|---|---|---|
| Sample | coating cycles | appearance (min) |
| Brevoxyl ™ 8%^{(a)} | Commercial product | 3 |
| NeoBenz ™ 5.5%^{(b)} | Commercial product | 8 |
| SGT-V5, 20%^{(c)} | 5 | 16 |
| SGT-V10, 20%^{(c)} | 10 | 37 |
| SGT-V15, 20%^{(c)} | 15 | 108 |
| SGT-V20, 20%^{(c)} | 20 | 152 |

^{(a)}Commercial product containing 8% w/w BPO.
^{(b)}Commercial product containing 5.5% w/w BPO.
^{(c)}Containing 20% w/w BPO in the suspension.
All are normalized to 200 mg BPO in the test method.

### Example #3: Skin Irritation Study in Domestic Pigs with a Composition of the Invention

A 13 day cumulative skin irritation study in domestic pigs with 14 days recovery was conducted. The tested drug products included the following treatment groups:

DP #1: 4% E-BPO/0.3% ADPL: 4% E-BPO (silica encapsulated BPO) and 0.3% non-encapsulated adapalene

DP #2: 4% E-BPO/0.3% E-ADPL: 4% E-BPO (silica encapsulated BPO) and 0.3% encapsulated adapalene (silica encapsulated)

Epiduo® (Comparative product): 2.5% BPO and 0.1% adapalene

Untreated Comparative Group

The study was conducted in accordance with Standard Operating Procedures (SOPs) and in accordance with sponsor approved protocol. The study is approved by National Animal Care and Use Committee, and was conducted according to the Israeli Animal Welfare Act following the Guide for care and Use of Laboratory Animals (National-Research Council, 1996).

The study was conducted to evaluate and compare the cumulative skin irritation of two different drug products (DP) of the invention, and to compare the irritation potential of these DPs to a current commercial formulation (Epiduo® (Comparative product)) after 14 consecutive days of topical administration and naïve skin. Due to animal welfare concern in one animal, the treatment phase was terminated for all the animals on day 13. One treatment group of five female domestic pigs was administered the test articles via topical application to eight separate dose sites, once a day for 13 consecutive days, at a fixed dose of 1 gr/site on the back (3×3 cm). An untreated dose site was maintained as the control site on the back and was treated in the same manner as the treated sites, except no test article was applied (untreated group). Following 13 days of administration, the animals were maintained for a 14-day recovery period.

Observations for morbidity, mortality, injury, food and water consumption were conducted and recorded twice daily for all animals. Clinical observations were conducted once daily on each day of dosing. The test sites were scored according to Draize score—(Draize J, Woodard G, Calvery H. 1944. Methods for the study of irritation and toxicity of substances applied topically to the skin and mucous membranes. J Pharm Exp Ther 82:377-390) for erythemaleschar and edema pretest and daily from day 0 (prior to dosing and on dosing days) through Day 26. Body weights were measured and recorded weekly. Clinical observation was conducted pretest and daily. At study termination, the animals were euthanized. There were no mortalities during the study. Clinical findings observed are typical for this strain and age of the animal.

The results from the cumulative irritation scores and observations are summarized as follows:

DP 4% E-BPO/0.3% ADPL:

Very slight to well-defined erythema was present in 3 animals beginning on Study Day 12, elevating to very slight to moderate erythema by Study Day 13 in all 4 animals, and resolved to very slight erythema by Study Day 14 during recovery in 3 out of the 4 animals. Very slight to moderate erythema was present in one animal from study day 14 until the end of the study (Study Day 26), and very slight erythema was present in another animal from Study Day 19 until Study Day 24. Very slight edema was present in day 13 in 2 of 4 animals.

### DP 4% E-BPO/0.3% E-ADPL:

Very slight to well-defined erythema was present in all 4 animals beginning on Study Day 12 only resolved to very slight erythema by Study Day 14. Very slight to well-defined erythema was present in 3 out of 4 animals on Study Day 18 to Study Day 21. No corresponding edema was noted.

### EpiDuo® Gel:

Very slight to severe erythema was noted in all animals beginning on Study Day 7 until Study Day 20 and was resolved to very slight to moderate erythema until the last day (Study Day 26). Very slight to slight edema was present on Study Day 11 and continued during most of the recovery period on 3 out of 4 animals.

### Untreated.

Untreated group demonstrated sporadic observation of very slight erythema during the recovery period in 2 animals. There was no edema.

### Conclusions:

Application sites most severely affected in terms of severity and incidence of dermal irritation scores were those treated with EpiDuo® gel. On sites treated with DPs of the invention, no substantial differences were observed during all dosing period in erythema score. Untreated group showed the lowest irritation values.

### Example #4. Preparation of Encapsulated ADPL (3.06% E-ADPL Water Suspension)

### a) Oil Phase

8.62 grams of Butylated hydroxyl toluene (BHT) and 45.9 grams of adapalene (ADPL) were mixed in 137.92 grams of Squalane. 86.16 grams of Tetraethoxysilane (TEOS) were added, and the resulted mixture was milled at 5000 rpm in a ball mill for 10 minutes with an upper propeller mixer at a speed of 250 rpm for 7 minutes, followed by 400 rpm for 3 minutes. 140.4 grams of milled adapalene in oil was aliquoted out and then heated to 60° C. 9.0 grams of Beeswax were added and melted in the oil phase.

### b) Water Phase

3.3 grams CTAC (Cetrimonium Chloride) were dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) was added to the water phase and homogenized at 4000 rpm for 1 minute. 17.9 grams of sodium silicate extra pure solution (28%) were added to the emulsion. The pH of the emulsion was adjusted to 4.0 using HCl 5N solution. Water was added to complete the total weight of the mixture to 650 grams. The suspension was then stirred for 17 hours at 25° C. for the TEOS hydrolysis to be completed. The composition of the final encapsulated ADPL water suspension product is shown in Table 2.

TABLE 2
Composition of the encapsulated ADPL 3.06% water suspension

| Ingredient | % of pure ingredient in the suspension |
|---|---|
| Beeswax | 1.15 |
| Squalane | 9.19 |
| TEOS | 5.74 |
| ADPL | 3.06 |
| Cetrimonium Chloride | 0.15 |
| Sodium hydroxide | 0.74 |
| Hydrochloric acid | 0.40 |
| Sterile Water for Irrigation | 79.56 |

### Example #5. Preparation of Encapsulated ADPL

Step 1: Milling: 75 g of adapalene are suspended in 250 g of 0.3% CTAC solution. The adapalene is milled using a stator rotor mixer (IKA DK 50.11 inline homogenizer at 15,000 rpm). The milling is stopped when the particle size distribution (PSD) of the suspension is d(0.9)≤20 μm. The temperature is kept below 30° C. at all times.

Step 2: Coating: during the coating procedure the suspension is stirred with a mechanical dissolver, 60 mm, at 500 rpm at all times. The pH of the milled adapalene suspension is corrected to about 4 using HCl 5N solution. A portion of 0.5 g of 15% sodium silicate solution (15% w/w as SiO₂) is added and the suspension is stirred for 5 min. A portion of 0.5 g of 3% Polyquatemium 7 is added and the suspension is stirred for 5 min. The pH is readjusted to about 4 using 5N HCl solution. This procedure is repeated 5-100 times in order to create a series of silica layers around adapalene having different thickness.

The aging step: The coated adapalene suspension at pH 4.5 is kept for aging at room temperature under gentle agitation for 24 hrs.

### Example #6. Preparation of Formulation of Clindamycin Phosphate (1.2%), Encansulated ADPL (0.15%) and Encansulated BPO (3%) in Emulsion (Formulation I)

Oil Phase: 720.0 of grams Cyclomethicone 5-N, 540.0 of grams Cetyl Alcohol, 360.0 grams Polyoxyl 100 Stearate, and 540.0 grams of Glyceryl Monosterate are mixed at 70° C.

Water phase: 18.0 grams of Ethylenediaminetetraacetate Disodium salt are dissolved in 6500 grams of water. 720.0 grams of glycerin (99.5%) are added to the solution. After the solution is heated to 70° C., 72.0 grams of Carbopol 980 NF are added and the resulting mixture is homogenized at 3300 rpm for 10 minutes to ensure that all materials are completely melted and dissolved. 76.5 grams of sodium hydroxide (20%) are then added and the mixture is stirred under high shear for 10 minutes at no less than 70° C.

The oil phase is added to the water phase under high shear at 78° C. and the resulting emulsion is homogenized at 3300 rpm for 10 minutes. 36.0 grams of Citric Acid and 3,576 grams of encapsulated BPO 15% water suspension made as described in Example 2 are mixed. The resulting mixture is added to the emulsion at 65° C. and mixed at 1400 rpm for 10 minutes. The emulsion is cooled to 35° C. and the pH of the emulsion is adjusted to 3.5 using HCl 5N solution. After 882.3 grams of encapsulated ADPL 3.06% water suspension made as described in Example 4 are added, the emulsion is stirred at 1400 rpm for 10 minutes. 216 grams of Clindamycin Phosphate are added slowly to the emulsion until they are completely dissolved. HCl 5N is added to adjust the pH to 4.5, and then water is added until the total weight of the emulsion reaches 18 kilograms. The composition of a formulation prepared with encapsulated adapalene described in example 4, is shown in Table 3.

TABLE 3
Composition of Formulation I

| | % of pure ingredient in the |
|---|---|
| Ingredient | composition |
| Polyquarternium-7 | 0.11 |
| Hydrochloric Acid | 0.25 |
| Citric Acid, Anhydrous | 0.26 |
| Lactic Acid | 0.13 |
| Silicon Dioxide | 0.77 |
| Sodium hydroxide | 0.05 |
| Cetrimonium Chloride | 0.06 |
| Hydrous Benzoyl Peroxide | 3.00 |
| Beeswax | 0.06 |
| Squalane | 0.42 |
| Ethanol (Alcohol) | 0.21 |
| ADPL | 0.15 |
| Clindamycin Phosphate | 1.20 |
| Glycerin | 4.00 |
| Polyoxyl 100 stearate | 2.00 |
| Cetyl alcohol | 3.00 |
| Cyclomethicone | 4.00 |
| Glyceryl monostearate | 3.00 |
| Edetate Disodium | 0.10 |
| Carbopol 980 | 0.40 |
| Sterile Water for Irrigation | 76.83 |

1. A composition comprising as active agents:
benzoyl peroxide (BPO) in an amount of at least 2% wt; and
adapalene in an amount of at least 0.1% wt;
wherein at least one of said active agents is encapsulated in microcapsules.

2. The composition according to claim 1, wherein each of said active agents are encapsulated in microcapsules.

3. The composition according to claim 1, wherein said microcapsule has an inorganic shell.

4. The composition according to claim 3, wherein said microcapsule has an inorganic oxide shell.

5. The composition according to claim 1, wherein said BPO is in the form of a solid particulate matter.

6. The composition according to claim 1, wherein said BPO is in an amount of at least 2.5% wt in the composition and/or said adapalene is in an amount of at least 0.15% wt in the composition.

7. The composition according to claim 6, wherein said benzoyl peroxide is an amount of 3% wt, and said adapalene is in an amount of 0.15%.

8. The composition according to claim 4, wherein said inorganic oxide shell is directly deposited on said at least one active agent.

9. The composition according to claim 1 further comprising at least one additional active agent.

10. The composition according to claim 9, wherein said at least one additional active agent is an antibiotic agent.

11. The composition according to claim 10, wherein said antibiotic agent is clindamycin phosphate.

12. The composition according to claim 11, wherein said clindamycin phosphate is in an amount of at 1.2% wt.

13. The composition according to claim 4, wherein said inorganic oxide shell is selected from silica, titania, alumina, zirconia, ZnO, and mixtures thereof.

14. The composition according to claim 13, wherein said inorganic oxide shell is silica.

15. The composition according to claim 1, suitable for topical administration.

16. The composition according to claim 1, wherein said microcapsule is prepared by deposition of inorganic oxide shell on the surface of the solid particulate matter.

17. The composition according to claim 1, wherein said microcapsule further comprises phase changing material in the core of said microcapsule.

18. A method for treating a surface condition in a subject comprising topically administering onto the surface a composition of claim 1.

19. The method according to claim 18, wherein said surface is skin or mucosal membrane.

20. The method according to claim 18, wherein said surface condition is selected from acne, rosacea, psoriasis, photoaging skin, hyperpigmented skin, mucosal infected areas, inflamed dermatitis, and combinations thereof.

21. A kit comprising: (a) a first composition comprising BPO in the amount of at least 2% wt as a first active agent; and (b) a second composition comprising adapalene in the amount of at least 0.1% wt as a second active agent; wherein at least one of said first and second active agents are coated by a shell.

22. The kit of claim 21, further comprising a third composition comprising antibiotics.

23. The kit of claim 22, wherein the antibiotics is clindamycin phosphate in an amount of 1.2% wt.

24. The kit according to claim 21, wherein said shell is an inorganic shell.

25. The kit according to claim 24, wherein said shell is an inorganic oxide shell.

26. The kit according to claim 21, further comprising instructions for use in the treatment of a disease or disorder selected from one or more of acne, rosacea psoriasis, photoaging skin, hyperpigmented skin, inflamed dermatitis, mucosal infected areas, the use comprising combining said first and said second composition for said treatment.

27. A composition comprising as active agents:
benzoyl peroxide (BPO) in an amount of at least 2% wt;
adapalene in an amount of at least 0.1% wt; and
clindamycin phosphate in an amount of 1.2% wt,
wherein at least one of said active agents is encapsulated in microcapsules.

28. The composition according to claim 27, wherein each of said active agents are encapsulated in microcapsules.

29. A composition according to claim 27, wherein said microcapsule has an inorganic shell.

30. A composition according to claim 27, wherein said microcapsule has an inorganic oxide shell.

31. The composition according to claim 27, wherein said BPO is in the form of a solid particulate matter.

32. The composition according to claim 27, wherein said BPO is in an amount of at least 2.5% wt in the composition and/or said adapalene is in an amount of at least 0.15% wt in the composition.

33. The composition according to claim 32, wherein said benzoyl peroxide is an amount of 3% wt, and said adapalene is in an amount of 0.15%.

34. The composition according to claim 30, wherein said inorganic oxide is selected from silica, titania alumina, zirconia, ZnO, and mixtures thereof.

35. The composition according to claim 34, wherein said inorganic oxide is silica.

36. The composition according to claim 27, suitable for topical administration.

37. The composition according to claim 27, wherein said microcapsule further comprises phase changing material in the core of said microcapsule.

38. A method for treating a surface condition in a subject comprising topically administering onto the surface a composition of claim 27.

39. The method according to claim 38, wherein said surface is skin or mucosal membrane.

40. The method according to claim 38, wherein said surface condition is selected from acne, rosacea, psoriasis, photoaging skin, hyperpigmented skin, mucosal infected areas, inflamed dermatitis, and combinations thereof.

41. A kit comprising: (a) a first composition comprising BPO in the amount of at least 2% wt as a first active agent; and (b) a second composition comprising adapalene in the amount of at least 0.1% wt as a second active agent; wherein at least one of said first and second active agents are coated by a shell.

42. The kit according to claim 41, wherein said shell is an inorganic oxide shell.
